(19) United States
(12) Patent Application Publication
(10) Pub. No.: US 2020/0113421 A1
(43) Pub. Date: Apr. 16, 2020

(54) Endoscope with Reusable Core

(71) Applicant: Canon U.S.A., Inc., Melville, NY (US)

(72) Inventor: Alexander Altshuler, Cambridge, MA (US)

(21) Appl. No.: 16/159,413

(22) Filed: Oct. 12, 2018

(51) Int. Cl. A61B 1/00; A61B 5/00; G02B 23/26; A61B 1/06; G01N 21/359

(52) U.S. Cl. CPC A61B 1/00142; A61B 5/0066; A61B 1/00167; G01B 9/02044; A61B 1/0676; G01N 21/359; A61B 5/6847; G02B 23/26

(57) ABSTRACT

Apparatus, methods, and systems for incorporating a disposable sheath into a medical device having a probe, wherein the disposable sheath is intended to cover and protect a probe of an endoscope, without having to dispose of the probe.

### FIELD OF THE DISCLOSURE

The present disclosure relates generally to apparatus and methods for endoscopy and, more specifically, methods and apparatus for incorporating a disposable sheath, intended to cover and protect a probe of an endoscope, without having to dispose of the probe.

### BACKGROUND OF THE DISCLOSURE

Medical probes have the ability to provide images from inside a patient's body. Considering the potential harm capable to the human body caused by the insertion of a foreign object, it is preferable that the probe be as small as possible. Additionally, the ability to provide images within small pathways such as vessels, ducts, incisions, gaps and cavities dictates the use of smaller probes. One particularly useful medical probe is the spectrally encoded endoscopy (“SEE”), which is a miniature endoscope that can conduct high-definition imaging through a sub-mm diameter probe. Spectrally encoded endoscopy (“SEE”) is a technique that uses wavelength to encode spatial information on a sample, thereby allowing high-resolution imaging to be conducted through small diameter endoscopic probes. SEE can be accomplished using a quasimonochromatic or broad bandwidth light input into a single optical fiber. At the distal end of the fiber, a diffractive or dispersive optic disperses the light across the sample, which is reflected and returns back through the optic and optical fiber. Light from the optical fiber is detected by a wavelength detecting apparatus, such as a spectrometer. By detecting the light intensity as a function of wavelength, the image may be reconstructed. SEE techniques have been described in, e.g., U.S. Pat. Nos. 7,843,572, 8,145,018, 6,341,036, 7,796,270 and U.S. Patent Publication Nos. 2008/0013960, 2011/0237892, and 2017/0168232, the contents of which are herein incorporated by reference, in their entirety.

Another exemplary use of a medical probe could be optical coherence tomography (“OCT”), which is an imaging technique that uses coherent light to capture micrometer-resolution, two- and three-dimensional images from within optical scattering media (e.g., biological tissue). Optical coherence tomography is based on low-coherence interferometry, typically employing near-infrared light. An OCT system comprises a light source, a reference arm, a sample arm, a deflected or deflecting section, a reference mirror (also referred to as a “reference reflection”, “reference reflector”, “partially reflecting mirror” and a “partial reflector”), and one or more detectors. A typical OCT system may include a patient interface device or unit (“PIU”) and a catheter, such that the and the OCT system may interact with a sample. The light source operates to produce a light to the deflecting section, which splits the light from the light source into a reference beam passing into the reference arm and a sample beam passing into the sample arm. The light source maybe, for example, a broad band light source with a short coherence length such as a Superluminescent light emitting diode (SLED), a tunable laser, white light source, UV light, Infrared light, visible light, or other light sources commonly used in OCTs. Both of the reference and sample beams may combine (or recombine) at the deflecting section and generate interference patterns. The output of the OCT system and/or the interferometer thereof is continuously acquired with the one or more detectors, such as photodiodes, photomultiplier tubes, a linear CCD array, image sensor, CCD array, CMOS array or any type of a sensor system that provides information about the interference pattern. The one or more detectors measure the interference or interference patterns between the two radiation or light beams that are coupled, combined or recombined. In one or more embodiments, the reference and sample beams have traveled different optical path lengths such that a fringe effect is created and is measurable by the one or more detectors. Electrical analog signals obtained from the output of the system 100 and/or the interferometer thereof are converted to digital signals to be analyzed with a computer.

Exemplary disclosures of OCT probes may be found throughout the public art, including U.S. patent application Ser. No. 15/629,175, which is incorporated by reference herein, in its entirety.

Localized imaging medical procedures are becoming more and more prevalent diagnostic tool as of late. Making the portion of a probe entering or contacting a patient's body disposable is often the preferred solution to avoid cross contamination in medical procedures. For complex imaging probes it is customary to use an outer sheath covering the probe, wherein the sheath that can be disposed at the end of procedure, while saving the expensive imaging portion of the probe. However, with probe sizes continuously shrinking it is getting more and more problematic for a physicians or medical technicians to attach (usually by threading) these tiny and fragile probes, especially submillimeter diameter flexible probes, into sheaths each time before procedures.

The widely accepted solution to this challenge is to dispose the whole assembly, including the inner portion of the probe, after each use. That makes the disposable probe and the procedure incorporating this imaging probe much more expensive, and the disposable probe connection to the imaging device more complicated than needed.

Accordingly, it would be particularly beneficial to disclose methods and apparatus wherein a probe construction allows for simple change of the outer sheath without relying on medical practitioners to perform this task correctly. This in turn would significantly reduce incidents of damage to the fragile probes, and in fact eliminate the need for contact with the probe while incorporating a sheath.

### SUMMARY

Thus, to address such exemplary needs in the industry, the presently disclosed apparatus, systems, and methods teach a probe configured to transmit an image, an imaging system attached to the probe and configured to receive the transmitted image from the probe, a reciprocator in communication with the probe and configured to advance and retract the probe a distance, as well as a docking station, and a disposable sheath removably attached to the docking station. Wherein the probe is configured to advance into the disposable sheath when the sheath is attached to the docking station, and wherein the probe is configured to retract into the docking station such that the disposable sheath may be removed from the docking station.

In further embodiments, the disposable sheath is removably attached to the docking station. The attachment means may be selected from a variety of existing known fastening methods, including, a bayonet connection, a rotating fitment, a compression fitting, and other non-permanent joints for mechanically affixing two object together.

Additional embodiments may further comprise an accumulator configured to accept at least of portion of the core as the core is being retracted.

Furthermore, the accumulator may comprise a substantially linear core storage portion, and/or a substantially cylindrical core storage portion.

In yet additional embodiments, the core is configured to rotate relative to the disposable sheath.

The subject disclosure further teaches a method for imaging a subject comprising the steps of providing a probe configured to transmit an image, followed by providing a imaging system in communication with the probe and configured to receive the transmitted image from the probe, thereafter providing a reciprocator in communication with the probe and configured to advance and retract the probe a distance, then providing a disposable sheath removably attached to the docking station, and attaching the disposable sheath to the docking station. In utilizing the probe and accompanying elements, the probe is advanced through the disposable sheath in preparation for capturing an image and transmitting the image to the imaging system; and thereafter the probe is retracted through the disposable sheath in preparation for removing the sheath without contacting the probe.

In additional embodiments, the disposable sheath is removably attached to the docking station.

In various embodiments, an accumulator is provided and configured to accept at least of portion of the core as the core is being retracted.

In yet additional embodiments, the accumulator may comprise a substantially linear core storage portion and/or a substantially cylindrical core storage portion.

In yet additional embodiments, the core may be configured to rotate relative to the disposable sheath.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 depicts a side perspective view of a medical imaging device, according to one or more embodiments of the present subject matter.

FIG. 2 provides a side perspective view of a medical imaging device, according to one or more embodiments of the present subject matter.

FIG. 3 is a side perspective view of a medical imaging device, according to one or more embodiments of the present subject matter.

FIG. 4 depicts a side perspective view of a medical imaging device, according to one or more embodiments of the present subject matter.

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

The present disclosure details a medical device having a probe construction which allows for simple change of the outer sheath of the probe without relying on medical practitioners to perform this task correctly.

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description and/or illustration to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated. The term “position” or “positioning” should be understood as including both spatial position and angular orientation.

Some embodiments of the present invention may be practiced in conjunction with a computer system that includes, in general, one or a plurality of processors for processing information and instructions, RAM, for storing information and instructions, ROM, for storing static information and instructions, a data storage device such as a magnetic or optical disk and disk drive for storing information and instructions, (e.g., an MRI image) an optional user output device such as a display device (e.g., a monitor) for displaying information to the computer user, and an optional user input device.

As will be appreciated by those skilled in the art, some aspects of the disclosure may be embodied, at least in part, as a computer program product embodied in any tangible medium of expression having computer-usable program code stored therein. For example, some aspects described below with reference to flowchart illustrations and/or block diagrams of methods, apparatus (systems) and computer program products can be implemented by computer program instructions. The computer program instructions may be stored in computer-readable media that can direct a computer or other programmable data processing apparatus to function in a particular manner, such that the instructions stored in the computer-readable media constitute an article of manufacture including instructions and processes which implement the function/act/step specified in the flowchart and/or block diagram.

In the following description, reference is made to the accompanying drawings which are illustrations of embodiments in which the disclosed invention may be practiced. It is to be understood, however, that those skilled in the art may develop other structural and functional modifications without departing from the novelty and scope of the instant disclosure.

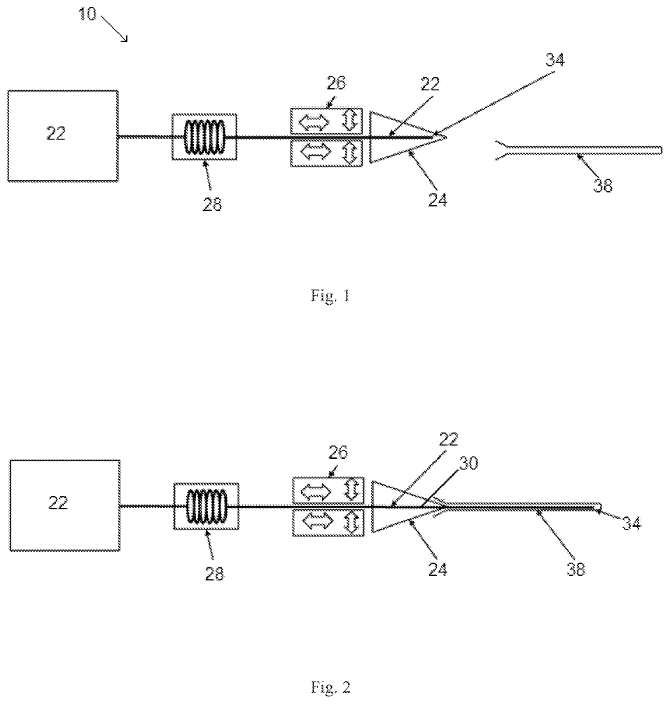

FIGS. 1 and 2 depict side perspective views of a medical imaging system 10, according to one or more embodiments of the present subject matter. Both Figures comprise an imaging system 10 in communication with a probe core 22, a docking station 24, a core translation mechanism 26 (also referred to as a “reciprocator”) and a core accumulator 28. In most imaging systems 10 the probe core 22 must be rotating to obtain proper images of the surrounding specimen. For optical fiber based systems, such as OCT and SEE, the probe core 22 usually comprises an optical fiber 30 inserted into a flexible torque transmitting drive shaft 32 with imaging optics 36 at the distal end 34 of the probe core 22. This fragile and function distal imaging optics 36 should be protected from particulates, rough handling, and contamination at all times. For this purpose, in idle system state, between imaging sessions, the probe core 22 is completely drawn inside docking station 24 by the reciprocator 26. The resulting excess probe core 22 is taken up by core accumulator 28.

When the disposable sheath 38 is attached to the docking station 24, the probe core 22 may be mechanically advanced from the docking station 24 into the disposable sheath 38 to position the medical imaging device 10 into the imaging state (FIG. 2). After use the probe core 22 is retracted into the docking station 24, allowing the disposable sheath 38 to be removed and disposed of without contact with the delicate probe core 22 and other components (FIG. 1). Thus, the probe core 22 will always be protected from rough handling and damage.

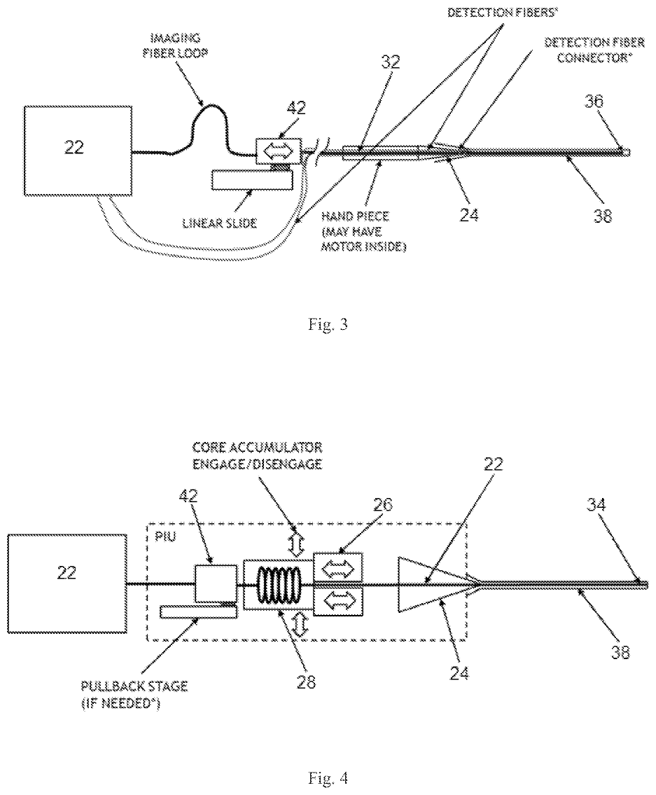

In various embodiments, where the length of the excess probe core 22 to be stored in the reciprocator 26 and/or accumulator 28 may have different designs, one embodiment of the subject disclosure intended for shorter probe lengths (FIG. 3) is comprised of a linear stage 40 moving the entire rotary joint 42 at a substantially straight withdrawing section of the same length as the required probe core 22 storage length. The withdrawing portion of the probe core 22 should be rigid enough to be self-supporting when pulled out of the disposable sheath 38. Preferably, the withdrawing portion would be made of hypodermic or similar tubing for added rigidity and security.

While the embodiments described above present various elements and/or methods of implementation, the disclosures all require the straight portion of the probe core 22 needed to be withdrawn to fit inside a docking station 24. As this may be impractical for the large probe lengths, another embodiment is herein disclosed/employed, as provided in FIG. 4.

The embodiment provided in FIG. 4 may be used, for example, in a cardiovascular OCT imaging requiring relatively long probes in a range of 500 mm to 2000 mm, and additional pullback motion of the imaging system 20 for longitudinal imaging of a blood vessel. In this embodiment a barrel type accumulator 28, adapted to receive the probe core 22 as it is being wound either on the inside or on the outside of a cylindrical surface, is connected to the spinning portion of the rotary joint 42. The core translation mechanism 26 takes advantage of the helical winding of the common design of the probe core 22 drive shaft and is comprised of a split lead screw nut adapted to the diameter and pitch of the drive shaft. Thus, with the nut engaged the probe core 22 will move distally or proximally depending of its rotational direction.

When accumulator fill mechanism is engaged the probe core translation mechanism 26 starts pulling the probe core 22 in the proximal direction from the probe core 22 into the accumulator 28 where it is wound on the cylindrical surface. After this process is finished the distal end of the probe core 22 is completely withdrawn into the docking station 24 and the disposable sheath 38 may be safely disconnected and discarded without contacting the probe core 22. After a new disposable sheath 38 is installed the core translation mechanism 26 may be engaged, in the opposite direction, to advance the probe core 22 by unwinding the accumulator 28 thus advancing the distal end of the probe core 22 through and towards the distal end of the sheath. Finally, when the accumulator 28 has sufficiently advanced the probe core 22 into the disposable sheath 38, the process stops and the system goes into the imaging state.

1. An apparatus comprising:
a probe configured to transmit a light;
a reciprocator in communication with the probe and configured to advance and retract the probe a distance;
a docking station; and
a disposable sheath removably attached to the docking station,
wherein the probe is configured to advance into the disposable sheath when the sheath is attached to the docking station, and
wherein the probe is configured to be retractable into the docking station such that the probe is recessed in the docking station.

2. The apparatus of claim 1, wherein the disposable sheath is removably attached to the docking station by a fastener.

3. The apparatus of claim 1, further comprising an accumulator configured to accept at least of portion of the probe as the probe is being retracted.

4. The apparatus according to claim 3, wherein the accumulator is configured to store a portion of the probe in an at least a partial loop.

5. The apparatus according to claim 3, wherein the accumulator comprises a substantially cylindrical probe storage portion.

6. The apparatus of claim 1, wherein the probe is configured to rotate relative to the disposable sheath.

7. The apparatus of claim 1, further comprising an imaging system attached to the probe and configured to receive the transmitted light from the probe and construct an image based on the light.

8. The apparatus of claim 1, wherein the probe is recessed such that the probe is prevented from contacting the disposable sheath.

9. A medical imaging apparatus comprising:
a probe configured to transmit a light;
an imaging system attached to the probe and configured to receive the transmitted light from the probe and construct an image based at least partially on the light;
a reciprocator in communication with the probe and configured to advance and retract the probe a distance;
a docking station; and
a disposable sheath removably attached to the docking station,
wherein the probe is configured to advance into the disposable sheath when the sheath is attached to the docking station, and
wherein the probe is configured to be retractable into the docking station such that the probe is recessed in the docking station.

10. A method for imaging a subject comprising the steps of:
providing a probe configured to transmit a light;
providing a reciprocator in communication with the probe and configured to advance and retract the probe a distance;
providing a disposable sheath removably attached to the docking station;
attaching the disposable sheath to the docking station;
advancing the probe through the disposable sheath in preparation for capturing the light and transmitting the light; and
retracting the probe into the docking station.

11. The method of claim 10, wherein the disposable sheath is removably attached to the docking station by a fastener.

12. The method of claim 10, further providing an accumulator configured to accept at least of portion of the probe as the probe is being retracted.

13. The method according to claim 12, wherein the accumulator is configured to store a portion of the probe in an at least a partial loop.

14. The method according to claim 12, wherein the accumulator comprises a substantially cylindrical probe storage portion.

15. The method of claim 10, wherein the probe is configured to rotate relative to the disposable sheath.

16. The method of claim 10, further comprising providing a imaging system in communication with the probe and configured to receive the transmitted light from the probe and construct an image based at least partially on the light.

17. The method of claim 10, wherein the probe is recessed such that the probe is prevented from contacting the disposable sheath.
